(19) United States
(12) Patent Application Publication
(10) Pub. No.: US 2020/0022774 A1
(43) Pub. Date: Jan. 23, 2020

(54) IMPLANTABLE MARKERS TO AID SURGICAL OPERATIONS

(71) Applicants: David Douglas, Winter Park, FL (US); Robert Douglas, Winter Park, FL (US); Kathleen Douglas, Winter Park, FL (US)

(72) Inventors: David Douglas, Winter Park, FL (US); Robert Douglas, Winter Park, FL (US); Kathleen Douglas, Winter Park, FL (US)

(21) Appl. No.: 16/509,592

(22) Filed: Jul. 12, 2019

Related U.S. Application Data
(60) Provisional application No. 62/700,473, filed on Jul. 19, 2018.

(51) Int. Cl. A61B 90/00; A61B 6/12; A61B 90/50

(52) U.S. Cl. CPC A61B 90/39; A61B 6/12; A61B 90/50; A61B 90/37; A61B 2090/3925; G06T 19/006; A61B 2090/374; A61B 2090/365; A61B 2090/376; A61B 2090/378; A61B 2090/392

(57) ABSTRACT

Deformation of mobile soft tissue is detected and represented in a three-dimensional medical image based on radiographically-detectable markers that are implanted in the tissue. Locations of the markers are co-registered with locations within the tissue. Sensed changes in relative locations of the markers are used to calculate relative changes in locations within the tissue due to deformation. The changes are used to calculate coordinated multi-voxel manipulations, such that a real-time volumetric medical imaging dataset is developed. By co-registering the operating room coordinate system and the volumetric medical imaging coordinate system, depth-3-dimensional augmented reality viewing of this real-time volumetric medical imaging dataset can be achieved, thereby improving the surgeon's understanding of underlying surgical anatomy and ultimately improving surgical outcomes.

### TECHNICAL FIELD

Aspects of this disclosure are generally related to localization of anatomic structures during surgery.

### BACKGROUND

To locate and excise a breast cancer mass during a surgical procedure the surgeon typically relies on a wire and hook that has been placed into the breast cancer lesion by a radiologist. Once placed, the wire is secured to the breast with tape and gauze packing material. Then, the patient is brought to the operating room where the surgeon uses the wire and hook as a guide to dissect down to the mass and excise the mass. Once the mass is excised, a surgical specimen is both radiographed and examined by a pathologist to confirm that the entirety of the cancerous tissue was removed.

There are some shortcomings with the current procedure. First, there exists the possibility of the wire getting dislodged during the transportation of the patient from the radiology department to the surgical operating table. Further, there exists the possibility of the wire and/or hook breaking. Further, once the wire is removed, the landmarks to denote the site of the breast tumor can be lost.

Augmented reality head display systems present a separate image to each eye to yield depth perception. Such systems are now gaining popularity in medicine. Some augmented reality systems are already FDA approved for enhancing surgical procedures since the systems provide both a real-world scene and a virtual image. In addition, augmented reality is being researched in diagnostic radiology with benefits including true 3D representation with depth perception, fly-through viewing and improved human machine interface (HMI). In fact, one study included augmented reality viewing of a breast cancer.

In presently known systems, especially in situations where tissues are mobile (e.g., breast tissues), there is limited ability for the augmented reality display of the diagnostic radiology images to be registered to the surgical anatomy. The breast tissue and breast lesion can move left/right, up/down, rotate, or change shape. A more accurate means for registering surgical anatomy to diagnostic radiological imaging would therefore have utility.

### SUMMARY

All examples, aspects and features mentioned in this document can be combined in any technically possible way.

In some implementations an apparatus comprises: a plurality of radiographically-detectable markers that are implanted in an anatomical structure; a radiographic scanner that detects the markers in the anatomical structure and generates two-dimensional scans of the anatomical structure; and an image processor that: uses the two-dimensional scans to co-register the detected markers with the anatomical structure by calculating a location of each detected marker relative to a location within the anatomical structure; generates a three-dimensional representation of the anatomical structure based on the two-dimensional scans; and adjusts the three-dimensional representation of the anatomical structure based on change in location of at least one of the detected markers relative to other ones of the detected markers as indicated in successive two-dimensional scans of the anatomical structure. In some implementations the markers comprise an emitter of electromagnetic energy. In some implementations at least one of the markers comprises a sensor. In some implementations each of the markers comprise a photon-emitting radiopharmaceutical. In some implementations at least some of the markers are interconnected. In some implementations at least some of the markers are attached to a single non-anatomical object. In some implementations each of the makers generates a uniquely identifiable output. In some implementations the image processor adjusts the three-dimensional representation of the anatomical structure by manipulating a plurality of voxels in a coordinated manner. In some implementations the image processor co-registers the location of each detected marker with an operating room coordinate system. In some implementations the image processor calculates a location of a first detected marker based on respective known locations of other ones of the detected markers. In some implementations the image processor adjusts the three-dimensional representation of the anatomical structure based on positional change of at least one of the detected markers relative to the other ones of the detected markers. In some implementations the image processor adjusts the three-dimensional representation of the anatomical structure based on orientational change of at least one of the detected markers relative to the other ones of the detected markers. In some implementations the image processor adjusts the three-dimensional representation of the anatomical structure based on configurational change of at least one of the detected markers relative to the other ones of the detected markers.

In some implementations a method comprises: implanting a plurality of radiographically-detectable markers in an anatomical structure; detecting the markers in the anatomical structure; representing the detected markers in two-dimensional scans of the anatomical structure; co-registering the detected markers with the anatomical structure by calculating a location of each detected marker relative to a location within the anatomical structure; generating a three-dimensional representation of the anatomical structure based on the two-dimensional scans; and adjusting the three-dimensional representation of the anatomical structure based on change in location of at least one of the detected markers relative to other ones of the detected markers as indicated in successive two-dimensional scans of the anatomical structure. Some implementations comprise the markers emitting electromagnetic energy. Some implementations comprise sensing at least one environmental condition of the anatomical structure with at least one of the markers. Some implementations comprise each of the markers using a radiopharmaceutical to emit photons. Some implementations comprise interconnecting at least some of the markers. Some implementations comprise attaching at least some of the markers to a single non-anatomical object. Some implementations comprise each of the makers generating a uniquely identifiable output. Some implementations comprise adjusting the three-dimensional representation of the anatomical structure by manipulating a plurality of voxels in a coordinated manner. Some implementations comprise co-registering the location of each detected marker with an operating room coordinate system. Some implementations comprise calculating a location of a first detected marker based on respective known locations of other ones of the detected markers. Some implementations comprise adjusting the three-dimensional representation of the anatomical structure based on positional change of at least one of the detected markers relative to the other ones of the detected markers. Some implementations comprise adjusting the three-dimensional representation of the anatomical structure based on orientational change of at least one of the detected markers relative to the other ones of the detected markers. Some implementations comprise adjusting the three-dimensional representation of the anatomical structure based on configurational change of at least one of the detected markers relative to the other ones of the detected markers.

In accordance with an aspect, a method comprises: creating a list of structures (e.g., anatomic features, medical devices, etc.) in which understanding the precise positioning would be beneficial for the upcoming operation; for each structure, create a list of types of sensory information (e.g., pressure, temperature, etc.) which would be beneficial to the operation; determining a practical set of locations for each item on the lists including the optimum number/type of sensors/emitters; performing placement of sensor(s)/emitter(s) to the list above (e.g., skin, subcutaneous sites, peri-tumoral sites, intra-tumoral sites, surgical devices, etc.); performing cross-sectional scan (e.g. CT, MRI, SPECT, PET, multi-modality, etc.) so that the newly placed emission-capable, radiologically-detectable sensor(s)/emitter(s) and the internal anatomy of interest (e.g., breast mass) are co-registered in the same volumetric dataset; performing co-registration of volumetric dataset and other selected objects for use in the operating room (e.g., surgeon's augmented reality headset, hand-held surgical device(s), electromagnetic detectors, intra-operative radiological equipment (e.g., orthogonal gamma cameras), any additional emission-capable sensor(s)/emitters, etc.) within the operating room into the Operating Room coordinate system (See, e.g. U.S. patent application Ser. No. 15/949,202, titled SMART OPERATING ROOM EQUIPPED WITH SMART SURGICAL DEVICES, which is incorporated by reference); initializing intraoperative continuous tracking of all structures tracked in the operating room. Surgeon wears the AR glasses, such that he/she can see the surgical anatomy of interest (e.g., breast mass) in the virtual image on the AR glasses; beginning surgery and noting that as the surgeon begins the procedure, the location of the surgical anatomy of interest will change (e.g., surgeon applies tension to the breast and the breast mass changes in orientation, position and configuration); using the real-time information (i.e., location, orientation, configuration, sensory data) from the sensor(s)/emitter(s) to model voxel manipulations (See, e.g. U.S. patent application Ser. No. 16/195,251, titled INTERACTIVE VOXEL MANIPULATION IN VOLUMETRIC MEDICAL IMAGING FOR VIRTUAL MOTION, DEFORMABLE TISSUE, AND VIRTUAL RADIOLOGICAL DISSECTION, which is incorporated by reference) within the 3D imaging dataset (e.g., surgeons AR glasses displays a virtual image consisting of the new modeled orientation, position and configuration of the breast mass and if desired, the emission-capable stereotactic markers could also be displayed). Real time surgeon viewing methods may include viewing through an Augmented Reality set of glasses (See, e.g. U.S. Pat. No. 8,384,771, titled METHOD AND APPARATUS FOR THREE DIMENSIONAL VIEWING OF IMAGES, which is incorporated by reference).

In accordance with an aspect, this method comprises sensor/emitter guided manipulation of voxel(s) location (i.e., x-coordinate, y-coordinate and z-coordinate) in the volumetric medical imaging dataset. As an example, during the surgery, the breast can change in position, orientation and configuration, which causes the implantable sensors/emitters to move into a new position/orientation/configuration. The detector system would detect the changes in the sensors/emitters and send this data to the program, which would perform changes in voxel location(s). The new voxel location(s) could be viewed by the surgeon via a virtual image on augmented reality glasses.

In accordance with an aspect, this method comprises sensor/emitter guided manipulation of voxel(s) orientation (i.e., roll, pitch and yaw) in the volumetric medical imaging dataset. As an example, during the surgery, the breast can change in position, orientation and configuration, which causes the implantable sensors/emitters to move into a new position/orientation/configuration. The detector system would detect the changes in the sensors/emitters and send this data to the program, which would perform changes in voxel orientation(s). The new voxel orientation(s) could be viewed by the surgeon via a virtual image on augmented reality glasses.

In accordance with an aspect, this method comprises sensor/emitter guided manipulation of voxel(s) size (i.e., volume) in the volumetric medical imaging dataset. As an example, during the surgery, the breast can change in position, orientation and configuration, which causes the implantable sensors/emitters to move into a new position/orientation/configuration. The detector system would detect the changes in the sensors/emitters and send this data to the program, which would perform changes in voxel size(s). The new voxel size(s) could be viewed by the surgeon via a virtual image on augmented reality glasses.

In accordance with an aspect, this method comprises sensor/emitter guided manipulation of voxel(s) configuration (e.g., cylindrical, cube, spherical, etc.) in the volumetric medical imaging dataset. As an example, during the surgery, the breast can change in position, orientation and configuration, which causes the implantable sensors/emitters to move into a new position/orientation/configuration. The detector system would detect the changes in the sensors/emitters and send this data to the program, which would perform changes in voxel configuration(s). The new voxel configuration(s) could be viewed by the surgeon via a virtual image on augmented reality glasses.

In accordance with an aspect, this method comprises sensor/emitter guided manipulation of voxel(s) internal property (e.g., Hounsfield Unit in CT, Intensity Unit in MRI, adding new color value, adding new biological-type tissue property, adding new chemical-type property, etc.) in the volumetric medical imaging dataset. As an example, during the surgery, the breast can change in position, orientation and configuration, which causes the implantable sensors/emitters to move into a new position/orientation/configuration. The detector system would detect the changes in the sensors/emitters and send this data to the program, which would perform change in voxel internal properties. The new voxel internal properties(s) could be viewed by the surgeon via a virtual image on augmented reality glasses.

In accordance with an aspect, this method comprises sensor/emitter guided voxel creation and insertion (e.g., invisible-type or air-type voxels inserted in the dissection pathway, tissue-type voxels, surgical instrumentation-type voxels, etc.) in the volumetric medical imaging dataset. As an example, during the surgery, the breast can change in position, orientation and configuration, which causes the implantable sensors/emitters to move into a new position/orientation/configuration. Sensors/emitters can also be placed on the skin surface or surgical instrumentation. The detector system would detect the changes in the sensors/emitters and send this data to the program, which would perform the insertion and creation of new voxels. The created and inserted voxels could be viewed by the surgeon via a virtual image on augmented reality glasses.

In accordance with an aspect, this method comprises sensor/emitter guided voxel elimination (i.e., removal from the volumetric medical imaging dataset). As an example, during the surgery, the surgeon could resect a portion of tissue containing sensors, which is removed from the body. The detector system would detect the changes in the sensors/emitters and send this data to the program, which would perform the removal of voxels from the medical imaging dataset. The void from the elimination of voxels would be noted by the surgeon via a virtual image on augmented reality glasses.

In accordance with an aspect, this method comprises sensor/emitter guided coordinated multi-voxel alterations including voxel manipulations (i.e., location, orientation, size, shape, internal property), voxel creation and insertions and voxel elimination. As an example, during the surgery, the surgeon could be simultaneously inserting a surgical instrument into the breast, removing a portion of breast tissue and deforming the breast tissue. Simultaneous voxel manipulations, additions and eliminations performed in real time would account for multiple simultaneous tasks.

In accordance with an aspect, an apparatus comprises: an implantable sensor/emitter with delivery system; a detector array; an instrument with an inertial navigation system (INS) to co-register the coordinate system used in the medical imaging dataset with the coordinate system used in the operating room; augmented reality glasses; an IO device; and, an image processor in communication with the IO device, the image processors comprising a program stored on computer-readable non-transitory media, the program comprising: creating a list of structures (e.g., anatomic features, medical devices, etc.) in which understanding the precise positioning would be beneficial for the upcoming operation; for each structure, create a list of types of sensory information (e.g., pressure, temperature, etc.) which would be beneficial to the operation; determining a practical set of locations for each item on the lists including the optimum number/type of sensors/emitters; performing placement of sensor(s)/emitter(s) to the list above (e.g., skin, subcutaneous sites, peri-tumoral sites, intra-tumoral sites, surgical devices, etc.); performing cross-sectional scan (e.g. CT, MRI, SPECT, PET, multi-modality, etc.) so that the newly placed emission-capable, radiologically-detectable sensor(s)/emitter(s) and the internal anatomy of interest (e.g., breast mass) are co-registered in the same volumetric dataset; performing co-registration of volumetric dataset and other selected objects for use in the operating room (e.g., surgeon's augmented reality headset, hand-held surgical device(s), electromagnetic detectors, intra-operative radiological equipment (e.g., orthogonal gamma cameras), any additional emission-capable sensor(s)/emitters, etc.) within the operating room into the Operating Room coordinate system; initializing intraoperative continuous tracking of all structures tracked in the operating room. Surgeon wears the AR glasses, such that he/she can see the surgical anatomy of interest (e.g., breast mass) in the virtual image on the AR glasses; beginning surgery and noting that as the surgeon begins the procedure, the location of the surgical anatomy of interest will change (e.g., surgeon applies tension to the breast and the breast mass changes in orientation, position and configuration); using the real-time information (i.e., location, orientation, configuration, sensory data) from the sensor(s)/emitter(s) to model voxel manipulations within the 3D imaging dataset (e.g., surgeon's AR glasses displays a virtual image consisting of the new modeled orientation, position and configuration of the breast mass and if desired, the emission-capable stereotactic markers could also be displayed). Real time surgeon viewing methods may include through an Augmented Reality set of glasses.

In accordance with an aspect this apparatus comprises sensor/emitter guided manipulation of voxel(s) location (i.e., x-coordinate, y-coordinate and z-coordinate) in the volumetric medical imaging dataset. As an example, during the surgery, the breast can change in position, orientation and configuration, which causes the implantable sensors/emitters to move into a new position/orientation/configuration. The detector system would detect the changes in the sensors/emitters and send this data to the program, which would perform changes in voxel location(s). The new voxel location(s) could be viewed by the surgeon via a virtual image on augmented reality glasses.

In accordance with an aspect this apparatus comprises sensor/emitter guided manipulation of voxel(s) orientation (i.e., roll, pitch and yaw) in the volumetric medical imaging dataset. As an example, during the surgery, the breast can change in position, orientation and configuration, which causes the implantable sensors/emitters to move into a new position/orientation/configuration. The detector system would detect the changes in the sensors/emitters and send this data to the program, which would perform changes in voxel orientation(s). The new voxel orientation(s) could be viewed by the surgeon via a virtual image on augmented reality glasses.

In accordance with an aspect this apparatus comprises sensor/emitter guided manipulation of voxel(s) size (i.e., volume) in the volumetric medical imaging dataset. As an example, during the surgery, the breast can change in position, orientation and configuration, which causes the implantable sensors/emitters to move into a new position/orientation/configuration. The detector system would detect the changes in the sensors/emitters and send this data to the program, which would perform changes in voxel size(s). The new voxel size(s) could be viewed by the surgeon via a virtual image on augmented reality glasses.

In accordance with an aspect this apparatus comprises sensor/emitter guided manipulation of voxel(s) configuration (e.g., cylindrical, cube, spherical, etc.) in the volumetric medical imaging dataset. As an example, during the surgery, the breast can change in position, orientation and configuration, which causes the implantable sensors/emitters to move into a new position/orientation/configuration. The detector system would detect the changes in the sensors/emitters and send this data to the program, which would perform changes in voxel configuration(s). The new voxel configuration(s) could be viewed by the surgeon via a virtual image on augmented reality glasses.

In accordance with an aspect this apparatus comprises sensor/emitter guided manipulation of voxel(s) internal property (e.g., Hounsfield Unit in CT, Intensity Unit in MRI, adding new color value, adding new biological-type tissue property, adding new chemical-type property, etc.) in the volumetric medical imaging dataset. As an example, during the surgery, the breast can change in position, orientation and configuration, which causes the implantable sensors/emitters to move into a new position/orientation/configuration. The detector system would detect the changes in the sensors/emitters and send this data to the program, which would perform change in voxel internal properties. The new voxel internal properties(s) could be viewed by the surgeon via a virtual image on augmented reality glasses.

In accordance with an aspect this apparatus comprises sensor/emitter guided voxel creation and insertion (e.g., invisible-type or air-type voxels inserted in the dissection pathway, tissue-type voxels, surgical instrumentation-type voxels, etc.) in the volumetric medical imaging dataset. As an example, during the surgery, the breast can change in position, orientation and configuration, which causes the implantable sensors/emitters to move into a new position/orientation/configuration. Sensors/emitters can also be placed on the skin surface or surgical instrumentation. The detector system would detect the changes in the sensors/emitters and send this data to the program, which would perform the insertion and creation of new voxels. The created and inserted voxels could be viewed by the surgeon via a virtual image on augmented reality glasses.

In accordance with an aspect this apparatus comprises sensor/emitter guided voxel elimination (i.e., removal from the volumetric medical imaging dataset). As an example, during the surgery, the surgeon could resect a portion of tissue containing sensors, which is removed from the body. The detector system would detect the changes in the sensors/emitters and send this data to the program, which would perform the removal of voxels from the medical imaging dataset. The void from the elimination of voxels would be noted by the surgeon via a virtual image on augmented reality glasses.

In accordance with an aspect, this apparatus comprises sensor/emitter guided coordinated multi-voxel alterations including voxel manipulations (i.e., location, orientation, size, shape, internal property), voxel creation and insertions and voxel elimination. As an example, during the surgery, the surgeon could be simultaneously inserting a surgical instrument into the breast, removing a portion of breast tissue and deforming the breast tissue. Simultaneous voxel manipulations, additions and eliminations performed in real time would account for multiple simultaneous tasks.

In accordance with an aspect, this apparatus comprises an implantable radiographically-detectable marker with embedded photon-emitting radionuclide optimized for continuous tracking by gamma cameras. The radiographically detectable marker could be made of high density (e.g., metal), which does not naturally occur in the human body and would therefore be easily recognized as a foreign body and could be used for the registration of the radioactive source to a pinpoint location within the body. Other parameters including size, shape, material would vary based on the type of surgery being performed. The activity of the source would decay in accordance with the half-life of the isotope. In the present disclosure, an example of a breast lumpectomy was discussed. In this case, the hook and wire could be one of the surgical devices embedded with a photon-emitting radionuclide to provide guidance to the surgeon throughout the lumpectomy.

In accordance with an aspect, this apparatus comprises implantable radiographically-detectable, electromagnetic radiation emitting optimized for continuous tracking by electromagnetic detectors. The radiographically detectable marker could be made of high density (e.g., metal), which does not naturally occur in the human body and would therefore be easily recognized as a foreign body and could be used for the registration of the electromagnetic energy source to a pinpoint location within the body. The electromagnetic energy emitted could vary in many parameters including frequency and intensity. Other parameters of the design including size, shape, material would vary based on the type of surgery being performed. In the present disclosure, an example of a breast lumpectomy is discussed. In this case, the hook and wire could be one of the surgical devices embedded with an electromagnetic energy emitter to provide guidance to the surgeon throughout the lumpectomy.

In accordance with an aspect, this apparatus comprises implantable radiographically-detectable sensors. The radiographically detectable marker could be made of high density (e.g., metal), which does not naturally occur in the human body and would therefore be easily recognized as a foreign body and could be used for the registration of the sensor to a pinpoint location within the body. The radiographically-detectable sensors could be paired with an electromagnetic energy emitter to communicated collected data by the sensor (e.g., temperature, pressure, chemical content, etc.). In this case, the hook and wire could be one of the surgical devices embedded with an sensor/emitter complex to provide guidance to the surgeon throughout the lumpectomy.

Finally, multiple pre-operative volumetric imaging examinations (e.g., CT or MRI) could be performed. This would improve the ability for assessment for voxel manipulation.

### BRIEF DESCRIPTION OF THE FIGURES

The patent or application file contains at least one drawing executed in color.

Copies of this patent or patent application publication with color drawing(s) will be provided by the Office upon request and payment of the necessary fee.

FIG. 1 illustrates an overview of the use of the implantable sensors/emitters to enhance surgery.

FIG. 2 illustrates an apparatus for implementing the process illustrated in FIG. 1.

FIGS. 3A and 3B illustrate a side-by-side comparison of the current surgical strategy for a lumpectomy with this patient's method/apparatus.

FIG. 4 illustrates important considerations for sensor/emitter design.

FIGS. 5A through 5F illustrate implementations of the electromagnetic emission subtype of sensory-capable, emission-capable, radiologically-detectable stereotactic emitters.

FIG. 6 illustrates the photon-emitting radiopharmaceutical subtype of emission-capable, radiologically-detectable stereotactic emitters.

FIG. 7 is a flow diagram that illustrates a method of placement of free-standing internal sensors/emitters and use of the delivery system.

FIG. 8 illustrates a method of placement of wire-type internal sensors/emitters.

FIG. 9 illustrates a method of performing real-time imaging of the photon-emitting radiopharmaceutical subtype of emission-capable radiologically-detectable stereotactic markers.

FIG. 10 illustrates a method of performing real-time imaging of the electromagnetic radiation subtype of sensor/emitter.

FIG. 11 illustrates a method for co-registering an operating room coordinate system with the volumetric medical imaging coordinate system.

FIG. 12 illustrates a scenario wherein multiple sensors/emitters have a location known by the cross-sectional imaging examination and one location of the sensors/emitters is not known by the cross-sectional imaging examination (e.g., sensor/emitter was placed after the cross-sectional imaging examination was performed) and a flow diagram of the primary method to assign coordinates within the medical imaging dataset to the emitters.

FIG. 13 illustrates an alternative method to determine the location of sensors/emitters placed after the cross-sectional imaging exam.

FIGS. 14A through 14D illustrate a wire equipped with multiple sensors/emitters changing configuration over multiple time points.

FIG. 15 illustrate the initial position of the sensors/emitters within the volumetric medical imaging dataset and how coordinate values would could change if the breast position is moved.

FIGS. 16A and 16B illustrate change in breast configuration with corresponding change in position of sensors/emitters and voxel manipulation to achieve change in configuration of the mass.

FIGS. 17A and 17B illustrate both a vertical and horizontal change in breast configuration with corresponding change in position of sensors/emitters and voxel manipulation to achieve change in configuration of the mass.

FIGS. 18A through 18C illustrates an example of voxel manipulation performed to better match the shape of the lesion.

FIG. 19 illustrates examples of a single voxel manipulation with change in voxel size, shape, position, orientation, or internal parameter.

### DETAILED DESCRIPTION

Some aspects, features and implementations described herein may include machines such as computers, electronic components, radiological components, optical components, and processes such as computer-implemented steps. It will be apparent to those of ordinary skill in the art that the computer-implemented steps may be stored as computer-executable instructions on a non-transitory computer-readable medium. Furthermore, it will be understood by those of ordinary skill in the art that the computer-executable instructions may be executed on a variety of tangible processor devices. For ease of exposition, not every step, device or component that may be part of a computer or data storage system is described herein. Those of ordinary skill in the art will recognize such steps, devices and components in view of the teachings of the present disclosure and the knowledge generally available to those of ordinary skill in the art. The corresponding machines and processes are therefore enabled and within the scope of the disclosure.

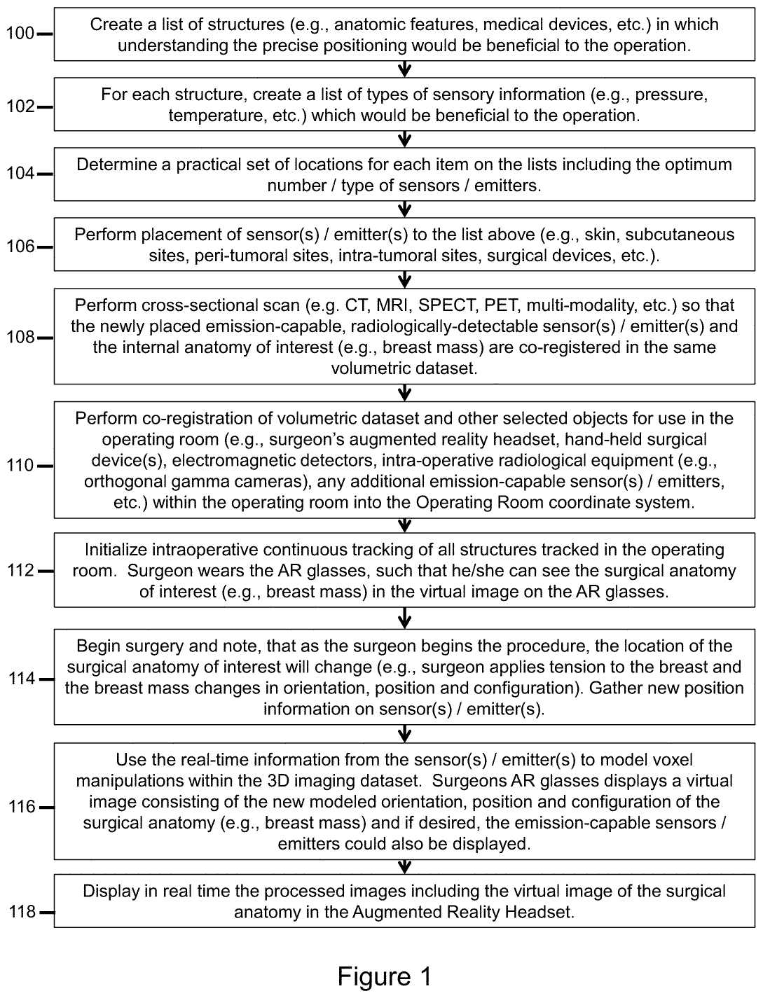

FIG. 1 illustrates an overview of the use of implantable markers (hereafter sensors/emitters) to enhance surgery. The types of surgeries where the sensors/emitters may yield the most benefit may include those on soft tissues, and more specifically mobile soft tissues, such as the breast. For this reason, various features will be described in the context of breast surgery. However, the features disclosed herein are not limited to breast surgery or mobile soft tissues and could be used in a wide variety of surgeries including but not limited to on the head, neck, face, chest, abdomen, pelvis and extremities. Several process steps are shown in FIG. 1. The first step 100 is to create a list of structures (e.g., anatomic features, medical devices, etc.) in which understanding the precise positioning would be beneficial to the operation. The next step 102 is to create, for each structure, a list of types of sensory information (e.g., pressure, temperature, etc.) which would be beneficial to the operation. The next step 104 is to determine a practical set of locations for each item on the list, the optimum number/type of sensors/emitters. The next step 106 is to perform placement of sensor(s)/emitter(s) at the list locations (e.g., skin, subcutaneous sites, peri-tumoral sites, intra-tumoral sites, surgical devices, etc.). The next step 108 is to perform cross-sectional scans (e.g. CT, MRI, SPECT, PET, multi-modality, etc.) so that the newly placed emission-capable, radiologically-detectable sensor(s)/emitter(s) and the internal anatomy of interest (e.g., breast mass) are co-registered in the same volumetric dataset. The next step 110 is to perform co-registration of the volumetric dataset and other selected objects for use in the operating room (e.g., surgeon's augmented reality headset, hand-held surgical device(s), electromagnetic detectors, intra-operative radiological equipment (e.g., orthogonal gamma cameras), any additional emission-capable sensor(s)/emitters, etc.) within the operating room into the Operating Room coordinate system (See. e.g. U.S. patent Application Ser. No,. 15/949,202). The next step 112 is to initialize intraoperative continuous tracking of all structures tracked in the operating room. The surgeon wears the AR glasses, such that he/she can see the surgical anatomy of interest (e.g., breast mass) in the virtual image on the AR glasses while looking down at the patient on the operating table. The next step 114 is to begin surgery. The location of the surgical anatomy of interest will change (e.g., surgeon applies tension to the breast and the breast mass changes in orientation, position and configuration) during the surgery. New position information on sensor(s)/emitter(s) is collected during the surgery, e.g. continuously. The next step 116 is to use the real-time information from the sensor(s)/emitter(s) to model voxel manipulations (See, e.g. U.S. Patent Application 62/695,868) within the 3D imaging dataset. The surgeon's AR glasses display a virtual image consisting of the new modeled orientation, position and configuration of the surgical anatomy (e.g., breast mass) and if desired, the emission-capable sensors/emitters could also be displayed. The final step 118 is to display in real time the processed images including the virtual image of the surgical anatomy in the Augmented Reality Headset, e.g. in accordance with U.S. Pat. No. 8,384,771.

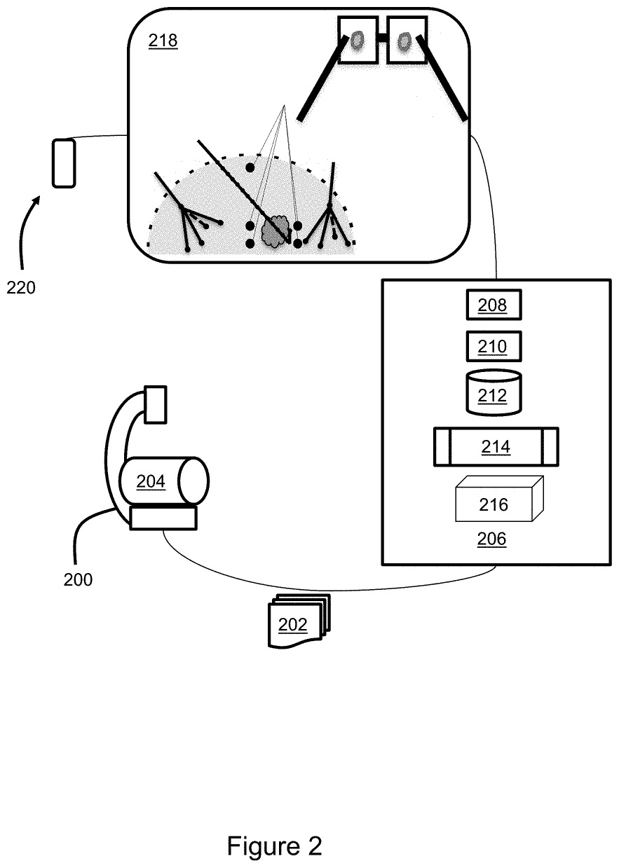

FIG. 2 illustrates an apparatus for implementing the process illustrated in FIG. 1. A radiologic imaging system 200 (e.g., X-ray, ultrasound, CT (computed Tomography), PET (Positron Emission Tomography), or MRI (Magnetic Resonance Imaging)) is used to generate medical images 202 of an anatomic structure 204 of interest (e.g., breast, breast mass, emitters/sensors in place along the skin surface, emitters/sensors internally without wire extending through the skin, emitters/sensors internally with wire extending through the skin, wire and hook, etc.). The medical images 202 are provided to an image processor 206, that includes processors 208 (e.g., CPUs and GPUs), volatile memory 210 (e.g., RAM), and non-volatile storage 212 (e.g. HDDs and SSDs). A program 214 running on the image processor implements one or more of the steps described in FIG. 1. Furthermore, subcomponent 216 represents electromagnetic detector set, nuclear imaging camera, radiological imaging device and subcomponents, e.g. as described in U.S. patent application Ser. No. 15/949,202, which together provide the apparatus for tracking the small implanted sensors/emitters in the operating area of interest. 3D medical images are generated from the 2D medical images and displayed on an IO device 218. The IO device may include a virtual or augmented reality headset, monitor, tablet computer, PDA (personal digital assistant), mobile phone, or any of a wide variety of devices, either alone or in combination. The IO device may include a touchscreen, and may accept input from external devices (represented by 220) such as a keyboard, mouse, and any of a wide variety of equipment for receiving various inputs. However, some or all the inputs could be automated, e.g. by the program 214.

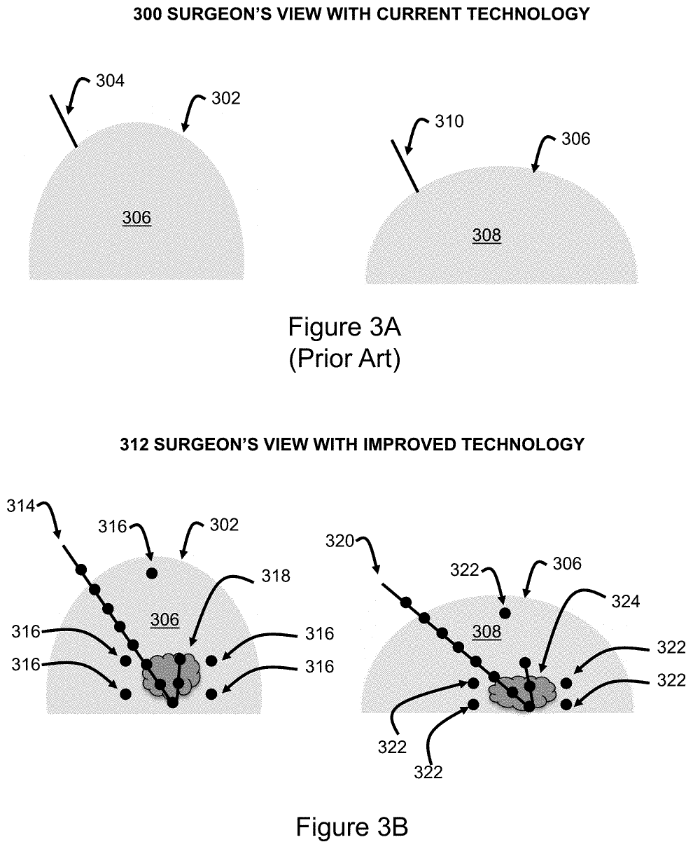

FIGS. 3A and 3B illustrate a side-by-side comparison of the current surgical strategy for a lumpectomy with one a strategy that benefits from the implantable sensors/emitters of the present disclosure. Prior to the surgery, the radiologist performs a mammogram to identify the mass or suspicious microcalcifications and performs needle localization with a wire and hook through the mass under image guidance. After the radiologist places the wire and hook through the breast mass, the radiologist secures the portion of the wire that is extending outward through the skin of the patient to the patient with gauze and tape. The patient is then brought to the operating room and general anesthesia is administered. The gauze over the wire is removed and the patient is prepped and draped in sterile fashion. Note that the current surgical strategy 300 is to resect the mass out of the breast, but the surgeon cannot see inside of the breast where the tumor is located. The surgeon can only see the skin of the breast 302 and the tip of the wire 304 protruding from the breast. The breast has an initial configuration/shape 306. At this point in time, the surgeon cannot see inside of the breast tissue, but only sees the trajectory of where the wire exits the body. The surgeon makes a skin incision and uses the wire as guidance to dissect down to the breast lesion and performs the resection. During the surgical dissection process, the deformable breast can assume an altered configuration 308 (e.g., change in size, shape, position). The skin surface may also change in configuration 306. Additionally, the wire exiting from the breast may also change in configuration 310. Still, the surgeon cannot see the new position, orientation and configuration of the breast mass. In contrast, the surgeon's view with the improved technology 312 is based on placement of a new type of wire and hook 314, which has emitters/sensors on it. Furthermore, the radiologist would place multiple additional sensors/emitters 316 into the region of the operation (e.g., peri-tumoral location). Then, the wire is secured with tape and gauze. Then, a cross-sectional imaging exam is performed (e.g., CT or MRI) so that the internal markers, the breast mass, and the breast are all co-registered on the same volumetric imaging dataset. Then, the patient is brought to the operating room, and general anesthesia is administered. The gauze over the wire is removed and the patient is prepped and draped in sterile fashion. At this point, the surgeon puts on his/her Augmented Reality glasses and can see a virtual image of the breast cancer mass 318. If desired, the surgeon can also see the virtual image of the emitters/sensors inside the body 316 and the wire and emitters 314. The surgeon may also see the skin surface 302. During the surgery, the breast mass changes its position since it is mobile soft tissue. During that process of the breast changing in position, orientation or configuration, the sensors/emitters also change in position, orientation or configuration. During the surgical dissection process, the deformable breast can assume an altered configuration 308 (e.g., change in size, shape, position). The skin surface may also change in configuration 306. Additionally, the wire may also change in configuration 320. Additionally, the sensors/emitters 322 may also change in position. Additionally, the surgeon can see that the skin surface changes in configuration. The detectors assess the new position (or other information) of the sensors/emitters 322 and/or wire with sensors/emitters 320. The detectors sense that the position of the emitted voxels has changed. Thus, the surgeon looking through the augmented reality glasses can see the new position and configuration of the breast mass 324. The computer program then performs voxel manipulation, e.g. in accordance with U.S. Patent Application 62/695,868. The resultant new position, orientation, and configuration of the mass from the voxel manipulation is then sent to the surgeon's augmented reality headset so he/she sees the change in the breast mass.

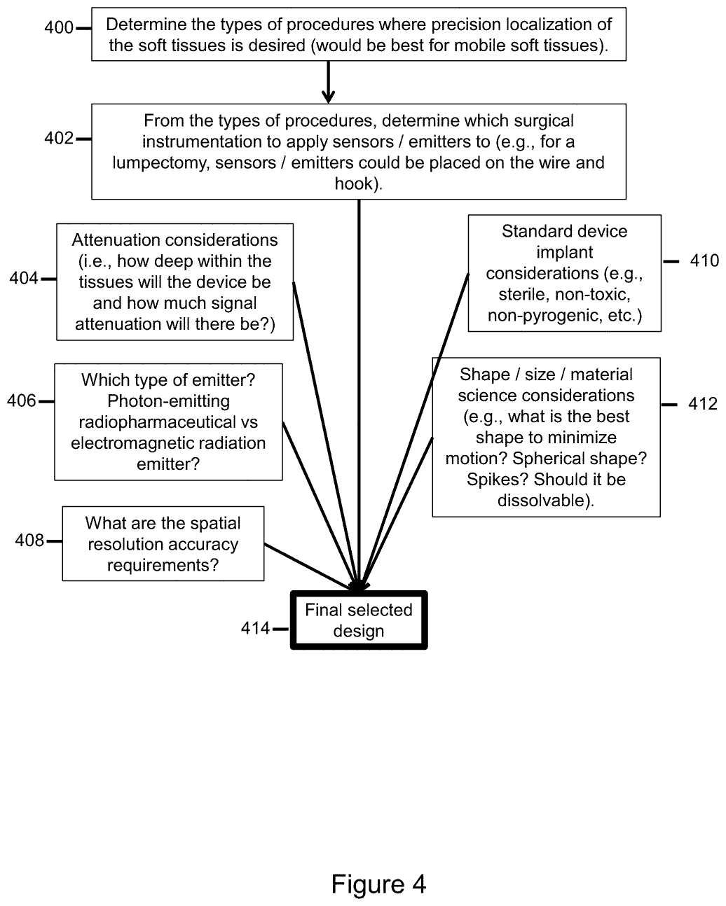

FIG. 4 illustrates a variety of considerations of sensor/emitter designs for different uses. The first consideration 400 is to determine the types of procedures where precision localization of the soft tissue types is desired. Various features described in the present disclosure may be useful for surgeries performed on soft tissues, and particularly mobile soft tissues. After determining the types of procedures, another consideration 402 is the standard surgical instrumentation used in those procedures and for which instruments precision localization would be important. The sensors/emitters will be applied to those locations. For example, during a lumpectomy, sensors/emitters could be placed on the wire and hook and several spots, which would allow the surgeon visualization through the virtual image projected on the augmented reality glasses where precisely the wire and hook travel through when underneath the skin. At any point in the procedure, re-registration could be performed of any sensor/emitter or other object registered during the procedure. Another consideration 404 for the sensor/emitter design is the amount of attenuation of signal/photons will undergo, which is a function of the type of tissues and the depth of tissues. As an example, a deeper placement would need to have a greater intensity of signal emitted in order to be received by the detectors. Another consideration 406 is the determination of which type of emitter to implement. Various types of emitters could be used, including a photon-emitting radiopharmaceutical (i.e., radioactive isotope) and an electromagnetic radiation emission type emitter. Since many patients undergo lymphoscintigraphy for sentinel lymph node mapping prior to the surgery and are already in the nuclear medicine department, it would be convenient for the patient to undergo placement of photon-emitting radionuclide markers/emitters. Various activity levels or isotopes could be used and coupled with appropriate collimators and orthogonal gamma cameras for localization in the operating room. Alternatively, electromagnetic radiation type emitters with small power sources (e.g., battery powered) and electromagnetic spectrum cameras coupled with the selected frequency could be used. Emitter fingerprinting could be through unique pulsation or frequency bands. An array of sensors placed into the body may yield benefit to the surgeon, such as temperature monitoring of breast tissues during free-flap reconstruction. Another consideration 408 is the spatial resolution accuracy of the emitter, such as within 1 mm, 0.5 mm or smaller. Standard device implant 410 considerations apply to all surgical devices that are placed in the body and include sterility, non-toxic, non-pyrogenic, and many others. Another consideration 412 is the shape of the emitter, such as determining the best shape to minimize motion through the soft tissues, such as whether a spherical shape or spiked margins for improved purchase to tissues would be beneficial. Another consideration is how to optimize the size of the emitter. Since many of the emitters should be radiographically detectable on the cross-sectional imaging exam, they would need to be a certain size in accordance with the spatial resolution of the scanner as well as having a distinguishing feature that separates it from the background soft tissues (e.g., a small metallic structure would have a Hounsfield unit that is different from the background soft tissues on a CT scan). This figure is not intended to include every possible consideration for every possible design but serves to highlight several design considerations that may be important.

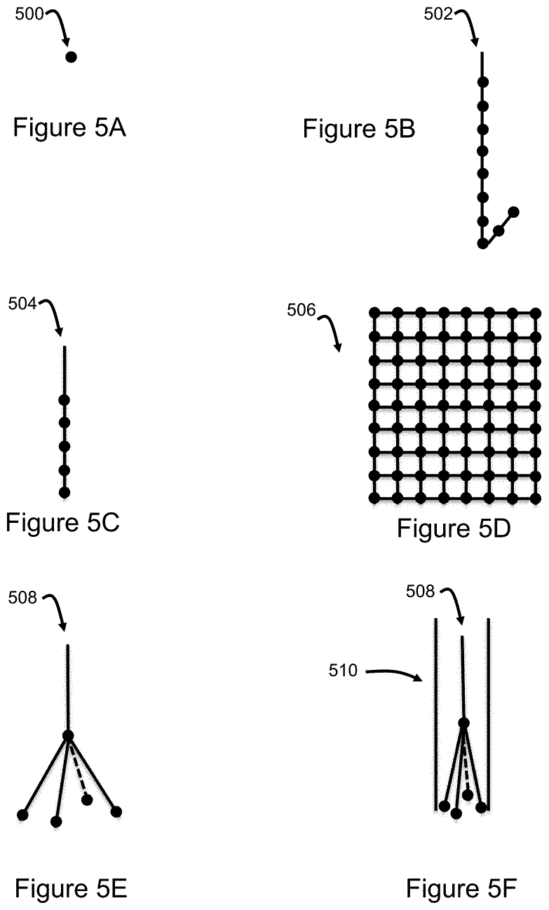

FIGS. 5A through 5F illustrate implementations of the electromagnetic emission subtype of sensory-capable, emission-capable, radiologically-detectable stereotactic emitters. The emitters may also be referred to as internal stereotactic markers because they function to provide location information. As shown in FIG. 5A, one or more individual emitters/sensors could be placed in a free-standing position 500 inside of the body without an attachment to other emitters/sensors, such as through needle-type delivery system with shape optimized to minimize motion. Each such individual emitter/sensor is independent of other emitter sensors. As shown in FIG. 5B, a group of sensors/emitters could be interconnected, such as by being attached to a common object or having interconnecting links. In the illustrated example the sensors/emitters are placed along the length of a wire and hook 502, commonly used in radiology departments for the needle localization procedure. The tip of the wire would typically extend outside of the skin. As shown in FIG. 5C, a group of the sensors/emitters could be interconnected by being placed along a wire 504, which could traverse the soft tissues. As shown in FIG. 5D, multiple sensors/emitters could be interconnected in a mesh or grid 506 that might be placed along the skin surface, likely with a sticky material so that the grid position relative to the immediately adjacent skin is fixed. As shown in FIG. 5E, interconnected sensors/emitters could be placed on a wire with a branching-type pattern 508. This would allow the placement of spatially separated sensors through a single skin puncture, rather than a linear fashion. As shown in FIG. 5F, the emitters on the wire and branch could be placed through a delivery sheath 510 where the branching wire 508 could collapse. It should be noted that wire-based emitters/sensors would potentially be easier to remove. Free-standing emitters/sensors may have a role but may be more difficult to remove. The emitters provide location information along with orientation information.

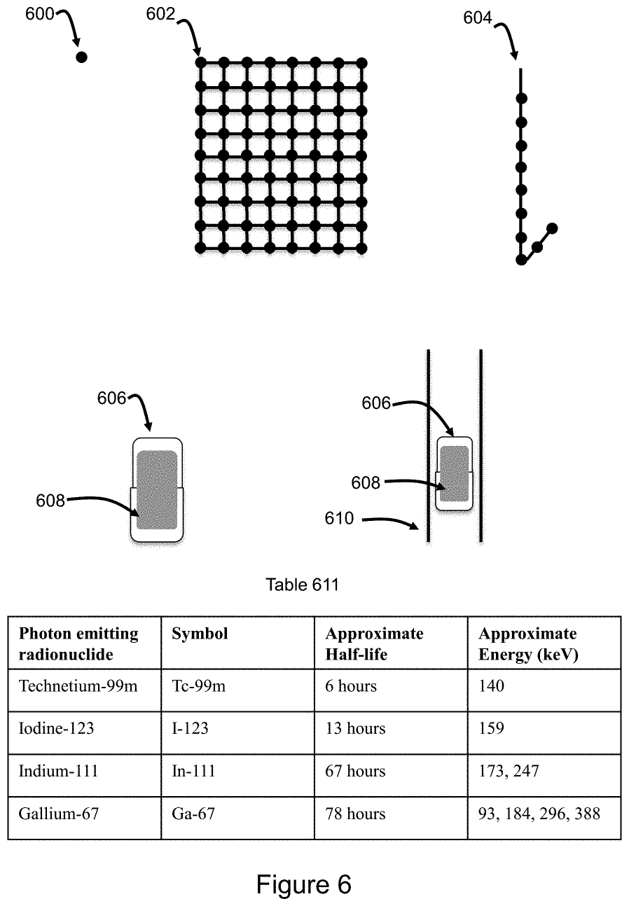

FIG. 6 illustrates the photon-emitting radiopharmaceutical subtype of emission-capable, radiologically-detectable stereotactic emitters. Note, in contrast to the electromagnetic subtype, this subtype would not be able to perform gathering of sensory data at the location of the marker. The radioisotope likely must be concentrated into a small volume. It should be appropriately sealed, so that it does not leak. Each marker should have its own unique signature, such that it can be better detected on gamma cameras, such as having a unique radioisotope or energy level. Each marker should also be radiographically-detectable via the cross-sectional imaging method used to build the 3D volumetric imaging dataset including the surgical anatomy and the emitters. Options to achieve this may include coupling a metallic density object with each emitter so that it can be seen on CT scan. Similar non-metallic markers could be used in MRI. Each marker should have a delivery system. The half-life should be optimized for imaging several hours after injection so when patient is in the OR. Several examples are shown. A first example is an individual free-standing emitter without attachment 600. A second example is interconnected emitters placed in a mesh-like pattern 602, such as for skin placement. A third example is interconnected emitters placed along a wire and hook 604. A fourth example is emitters 608 placed in a capsule 606. A fifth example of a delivery system includes a sheath 610. Table 611 lists commonly used radioisotopes in nuclear medicine including half-lives and energy levels. These radioisotopes or others could be used in as emitters.

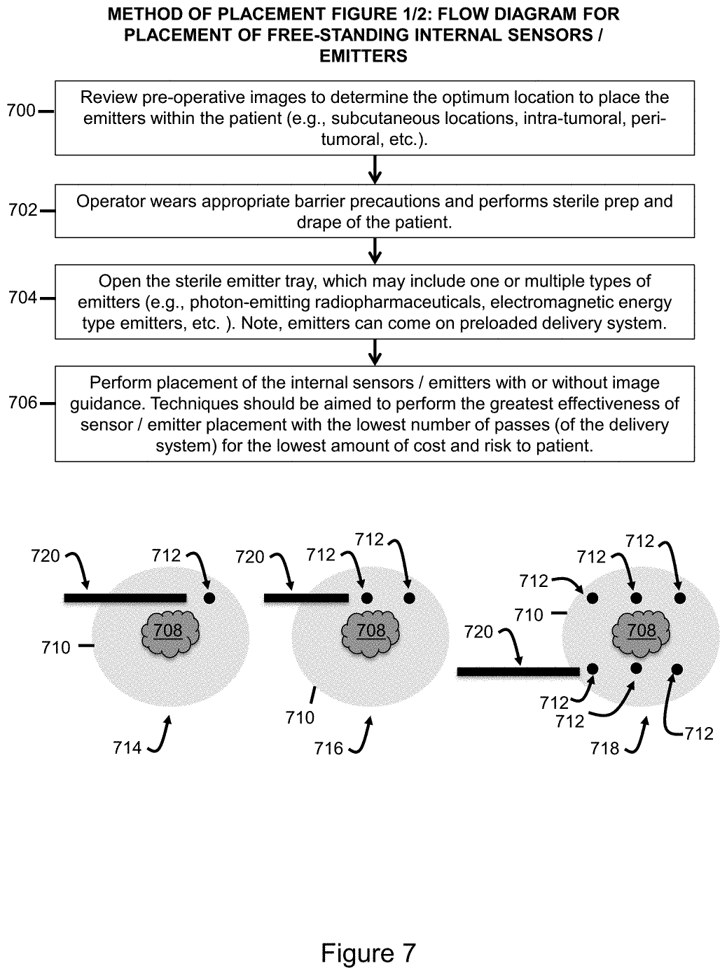

FIG. 7 is a flow diagram that illustrates a method of placement of free-standing internal sensors/emitters and use of the delivery system. Prior to any procedure, the prudent physician will review pre-operative imaging. In this scenario, the physician will focus on the surgical anatomy and where to optimally place the emitters within the patient (e.g., subcutaneous location, intra-tumoral location, peri-tumoral location, etc.). Additional pre-procedure items such as reviewing laboratory information, performing history and physical and informed consent are performed. Next, the operator will don the appropriate barrier precautions, perform a time out to confirm patient, procedure, equipment, etc. Next, the operator will open the sterile emitter tray, which may include one or more types of emitters, such as photon-emitting radiopharmaceuticals or electromagnetic radiation type emitters. Note, the emitters may come preloaded on a delivery system. Finally, the internal sensors/emitters are placed with or without image guidance (such as ultrasound). The techniques should be aimed to perform the greatest effectiveness of sensor/emitter placement with the lowest number of passes of the delivery system for the lowest amount of cost and lowest risk to the patient. In the illustration with a top-down view of the breast mass 708, the breast 710, the delivery system 720 is inserted through the skin in the desired location of the upcoming surgical procedure. As an example, this delivery system inserts three sensors/emitters 712 in each skin puncture. Two skin punctures are performed, and six sensors/emitters are placed. Time point #1 714, time point #2 716 and time point #3 718 are shown illustrating placement of six sensors/emitters 712.

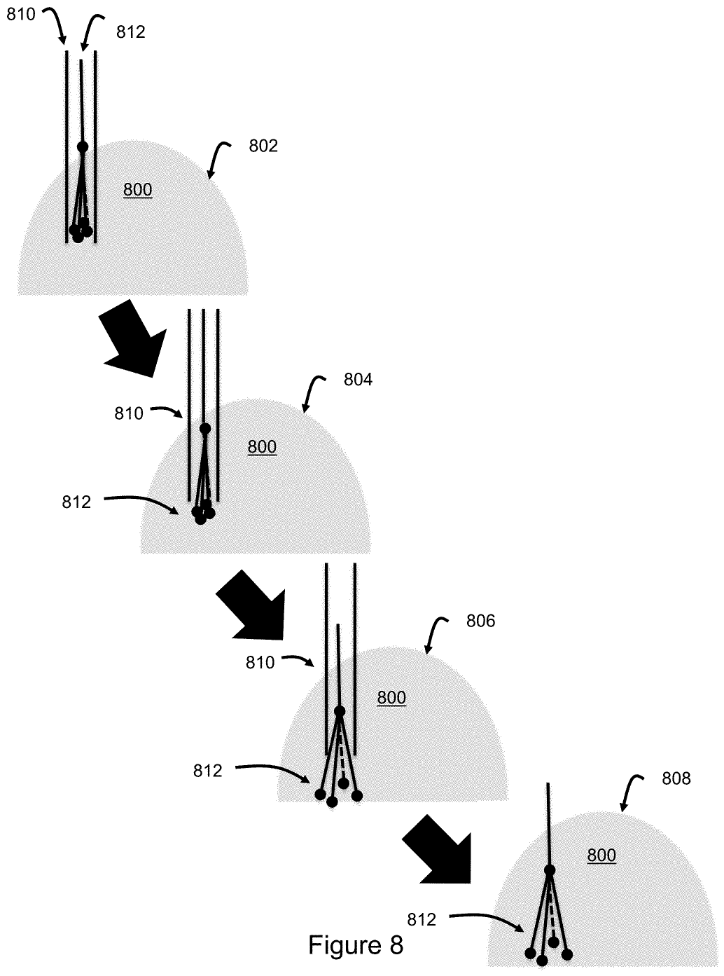

FIG. 8 illustrates a method of placement of wire-type internal sensors/emitters. The breast 800 is shown at four different time points. At the first time point 802, the sheath 810 containing the sensors/emitters on a wire apparatus 812 is advanced into the soft tissues. At the second time point 804, the sheath 810 is held in place and the wire apparatus 812 containing the sensors/emitters is advanced into the soft tissues. At the third time point 806, the sheath 810 is held in place and the wire apparatus 812 containing the sensors/emitters is advanced further into the soft tissues and the distal components begin spreading apart. At the fourth time point 808, the sheath is removed and the wire apparatus 812 containing the sensors/emitters remains within the soft tissues.

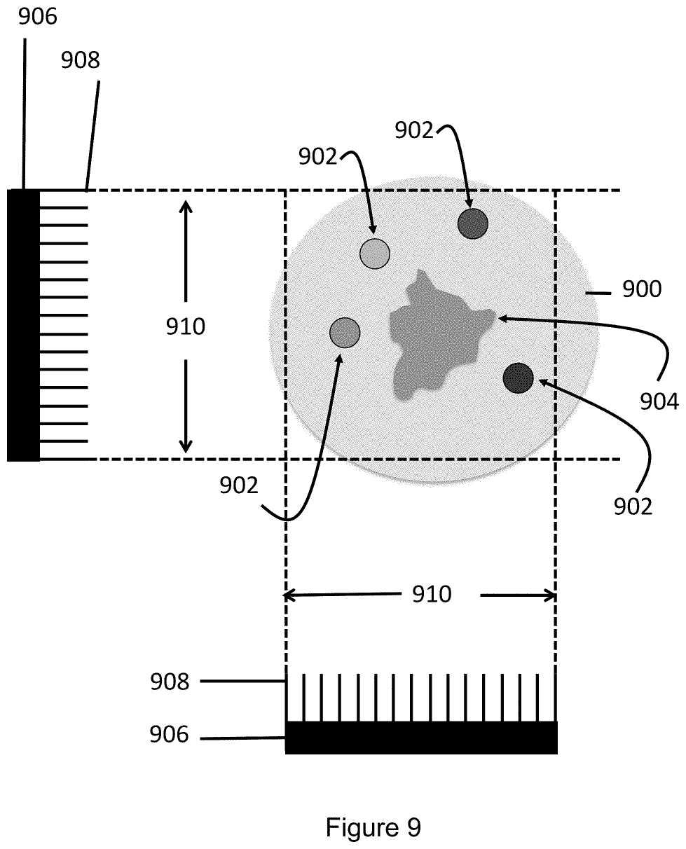

FIG. 9 illustrates a method of performing real-time imaging of the photon-emitting radiopharmaceutical subtype of emission-capable radiologically-detectable stereotactic markers. Two detector 906/collimator 908 units are oriented orthogonal to one another. Other placement positions are also possible. The type of collimator 908 illustrated is a parallel hole collimator, but other types of collimators are also possible. Thus, the field of view 910 is the same size as the detector. Four stereotactic photon-emitting radiopharmaceutical type stereotactic markers 902 are shown within the breast 900. Since each marker has its own unique signature, continuous imaging with back projection from the detectors is performed to determine the real-time localization of the emitters. Ultimately, described in more detail in other figures, the surgeon will be able to see the real time position of the breast mass in a virtual image on his/her augmented reality glasses.

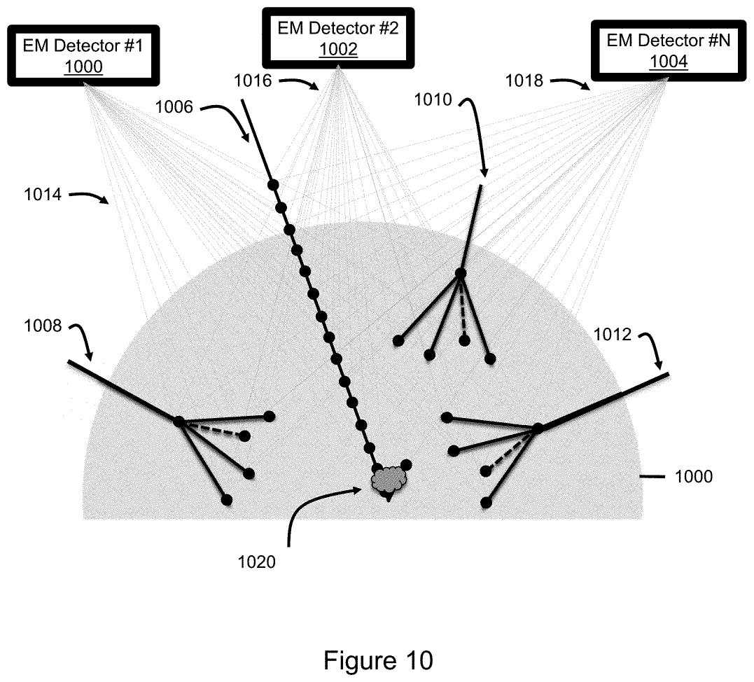

FIG. 10 illustrates a method of performing real-time imaging of the electromagnetic radiation subtype of sensor/emitter. In this illustration, several electromagnetic spectrum detectors are shown outside of the patient. As illustrated there is an electromagnetic (EM) detector #1 1000, EM detector #2 1002 and EM detector #N 1004. Three branching-wire type sensors/emitters 1008, 1010 and 1012 are shown with the sensors/emitters located inside the breast and the wire portion extending through the skin with tip outside of the breast. A wire and hook 1006 with many sensors/emitters is also present hooking through the breast mass. The EM signal travels from the sensors/emitters to the detectors and are illustrated as thin-dotted gray lines. The signal 1014 that travels to EM detector #1 1000 originates from the wire and hook 1006, first branching-wire type sensor/emitters 1008, second branching-wire type sensor/emitter 1010, third branching-type sensor/emitter 1012. The signal 1016 that travels to EM detector #2 1002 originates from the wire and hook 1006, first branching-wire type sensor/emitters 1008, second branching-wire type sensor/emitter 1010, third branching-type sensor/emitter 1012. Finally, the signal 1018 that travels to EM detector #N 1004 originates from the wire and hook 1006, first branching-wire type sensor/emitters 1008, second branching-wire type sensor/emitter 1010, third branching-type sensor/emitter 1012. The position, orientation and configuration of the mass 1020 within the breast 1000 can be inferred. This allows for real time monitoring of the internal tissues of the breast and provides data for voxel manipulation for the breast mass location/orientation/configuration for the surgeon's augmented reality headset.

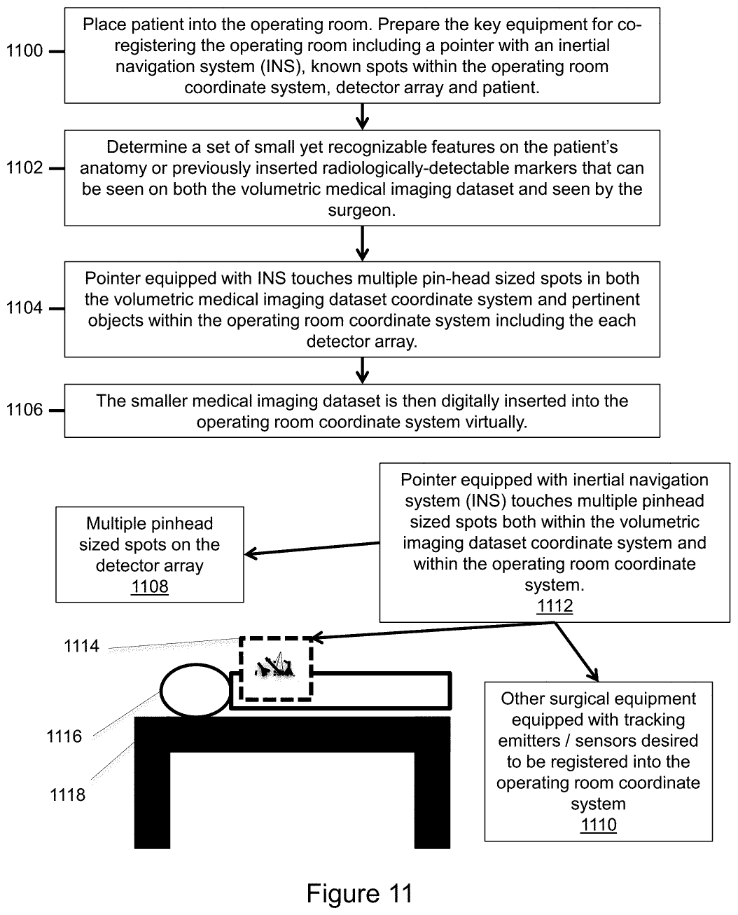

FIG. 11 illustrates a method for co-registering an operating room coordinate system with the volumetric medical imaging coordinate system. A first step 1100 is to place the patient in the operating room and prepare the equipment for co-registering the operating room including a pointer with an inertial navigation system (INS), known spots within the operating room coordinate system including the detector array, and known spots on the patient within the volumetric imaging dataset. The next step 1102 is to determine a set of small yet recognizable features on the patient's anatomy or previously inserted radiologically-detectable markers that can be seen on both the volumetric medical imaging dataset and seen by the surgeon. The next step 1104 is to use a pointer equipped with INS to touch-locate multiple pin-head sized spots in both the volumetric medical imaging dataset coordinate system and pertinent objects within the operating room coordinate system including the detector array. Note that one implementation would be to have the detector array on a stereotactic frame. The next step 1106 is for the smaller medical imaging dataset to be digitally inserted into the operating room coordinate system virtually. The pointer equipped with the inertial navigation system (INS) touches multiple pinhead sized spots both within the volumetric imaging dataset coordinate system and within the operating room coordinate system 1112 to include pinhead sized spots on the detector array 1108 and other surgical equipment with tracking emitters/sensors desired to be registered into the operating room coordinate system 1110. The field of view of volumetric medical imaging dataset 1114 is seen superimposed over the patient 1116 lying on the operating room table 1118.

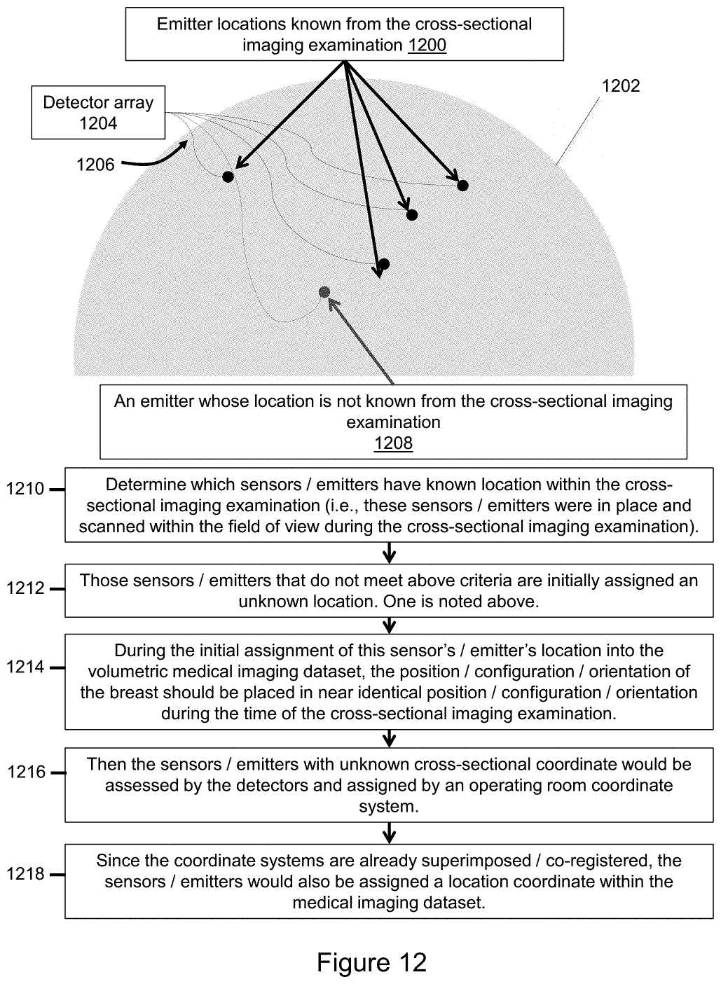

FIG. 12 illustrates a scenario wherein multiple sensors/emitters 1200 have known locations that were registered by a cross-sectional imaging examination and one of the sensors/emitters 1208 has an unknown location. The flow diagram illustrates a method to assign coordinates within the medical imaging dataset to such a sensor/

Attorney emitters. The coordinate positions of some emitters 1200 may be known because those emitters were inserted prior to the cross-sectional imaging examination and were within the field of view of the cross-sectional imaging examination. The coordinate positions relative to the nearby breast 1202 tissue accurately known. The location of sensor/emitter 1208 is not known. This may occur because the sensor/emitter was placed after the initial examination was performed or was outside of the scanned field of view). Thus, the location of sensor/emitter 1208 must be determined. Multiple methods can be performed to determine the locations of sensors/emitters. In the illustrated example an external detector is used for detecting the positions of multiple sensors/emitters with a known cross-sectional imaging coordinate system and known position within the operating room coordinate system, such that the two coordinate systems can be superimposed and co-registered. The first step 1210 is to determine which sensors/emitters have respective known locations within the cross-sectional imaging examination (i.e., these sensors/emitters were in place and scanned within the field of view during the cross-sectional imaging examination). The second step 1212 is for those sensors/emitters that do not meet above criteria to be initially assigned an unknown location. The third step 1214, during the initial assignment of this sensor's/emitter's location into the volumetric medical imaging dataset, is to recreate the position/configuration/orientation of the breast in a near identical position/configuration/orientation as at the time of the cross-sectional imaging examination. The fourth step 1216 is for the sensors/emitters with unknown cross-sectional coordinates to be assessed by the detectors and assigned by an operating room coordinate system. The fifth step 1218 is, since the coordinate systems are already superimposed/co-registered, the sensors/emitters are assigned a location coordinate within the medical imaging dataset. Ideally, during the initial assignment of sensor's/emitter's location into the volumetric medical imaging dataset, the position/configuration/orientation of the breast would be identical during the time of the cross-sectional imaging examination. Then the sensors/emitters with unknown cross-sectional coordinates would be assessed by the detectors and assigned by an operating room coordinate system. Since the coordinate systems are already superimposed/co-registered, the sensors/emitters would also be assigned a location coordinate within the medical imaging dataset.

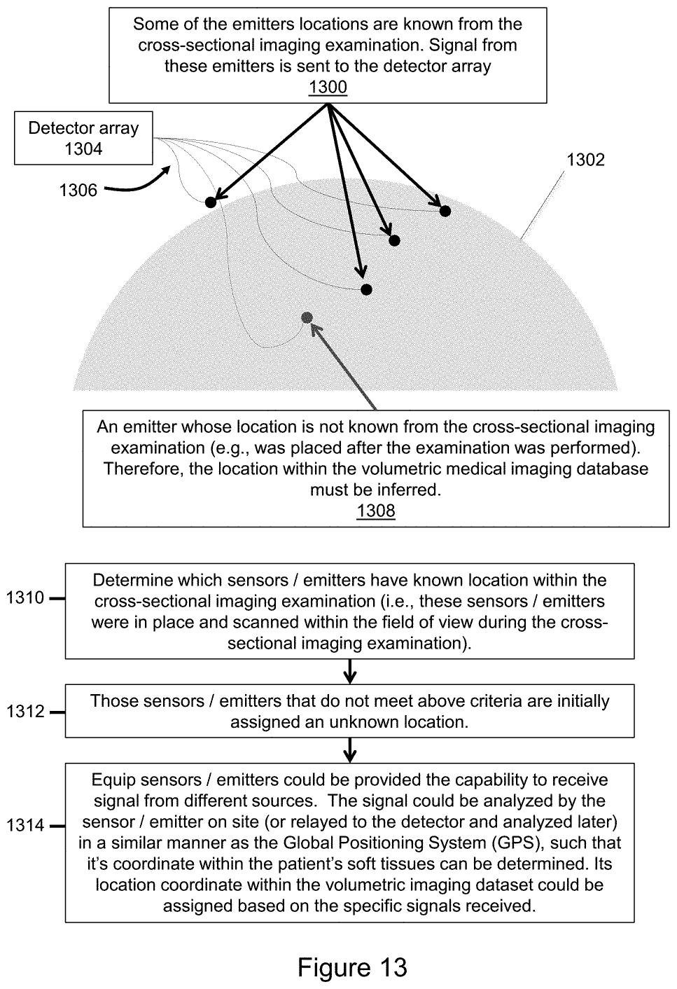

FIG. 13 illustrates an alternative method to determine the location of sensors/emitters placed after the cross-sectional imaging exam. Similar to FIG. 12, four emitters have locations known from the cross-sectional imaging examination 1300. The coordinate positions of these emitters as it relates to the nearby breast tissue is accurately known. The location of emitter 1308 is not known from the cross-sectional imaging exam (e.g., it was placed after the cross-sectional imaging exam was performed). Therefore, the coordinates within the volumetric imaging database must be inferred. EM signal 1306 goes from each of the emitters to the detector array 1304. In this alternative method, the sensors/ emitters could be provided the capability to receive signals from different sources. The first step 1310 is to determine which sensors/emitters have known locations within the cross-sectional imaging examination (i.e., these sensors/emitters were in place and scanned within the field of view during the cross-sectional imaging examination). The second step 1312 is for those sensors/emitters that do not meet above criteria to be initially assigned an unknown location. The third step 1314 is to utilize the sensors/emitters provided with the capability to receive signals from different sources. The signal could be analyzed by the sensor/emitter on site (or relayed to the detector and analyzed later) in a similar manner as the Global Positioning System (GPS), such that it's coordinate within the patient's soft tissues can be determined. Its location coordinates within the volumetric imaging dataset could be assigned based on the specific signals received. The signal could be analyzed by the sensor/emitter on site (or relayed to the detector and analyzed later) in a similar manner as the Global Positioning System (GPS), such that it's coordinate within the patient's soft tissues can be determined. Its location coordinate within the volumetric imaging dataset could be assigned based on the specific signals received.

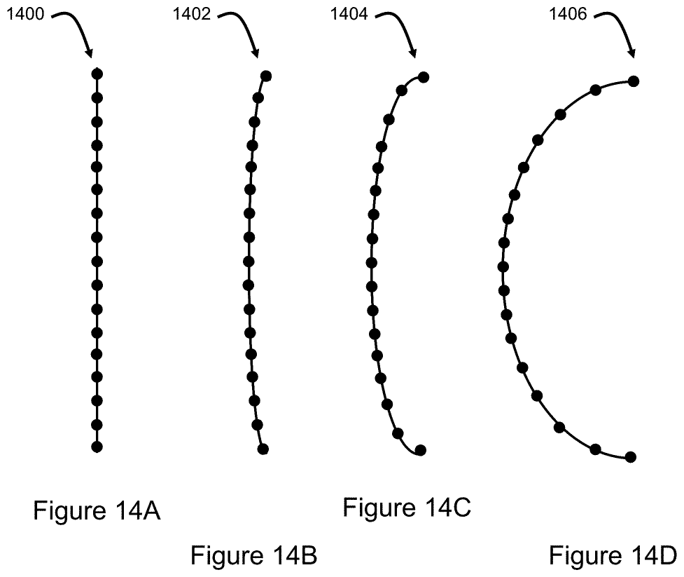

FIGS. 14A through 14D illustrate a wire equipped with multiple sensors/emitters changing configuration over time. As illustrated, the wire is equipped with multiple sensors/emitters. The sensors/emitters may change in location, orientation, position or other sensory information over time. At an initial time point shown in FIG. 14A, the sensors/emitters have a linear orientation 1400. At a subsequent time point shown in FIG. 14B, the sensors/emitters have a slightly curving orientation 1402. At a subsequent time point shown in FIG. 14C, the sensors/emitters have a slightly more curving orientation 1404. At a subsequent time point shown in FIG. 14D, the sensors/emitters have an even more curving orientation 1406. The sensors/emitters will relay data indicative of the changes of orientation to the detectors outside of the patient. The program will use this data to perform voxel manipulations in the volumetric imaging dataset to account for the ongoing changes in the mobile, soft tissue changes in the surgical operating bed.

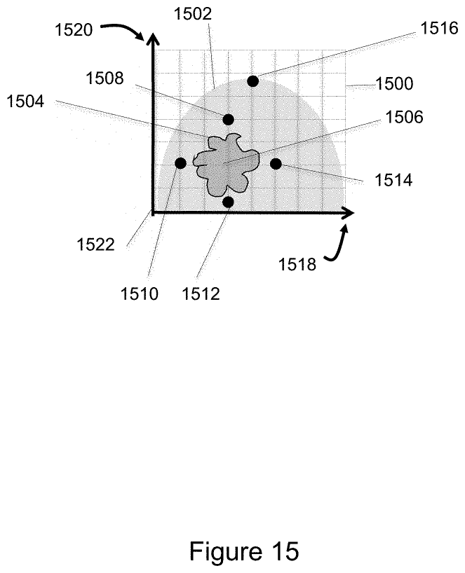

FIGS. 15A illustrate the initial position of the sensors/emitters with corresponding manipulation of breast voxels within the volumetric medical imaging dataset. A grid 1500 is shown over the breast 1502 and mass 1504. The voxel at the center of the breast mass 1506 is located at position (3.0, 2.0). For simplicity, 4 peri-tumoral sensors/emitters are shown with coordinates (3.0, 4.0) 1508, (1.0, 2.0) 1510, (3.0, 0.5) 1512 and (5.0, 2.0) 1514. Also noted is a sensor/emitter at the nipple with coordinate (4.0, 5.8) 1516. The x-axis 1518, y-axis 1520 and (0, 0) 1522 are also labeled. Note if a change in patient position occurs, such as placing an object underneath the patient's back and the whole body including the breast is raised. This could occur in situations such as placing a pad underneath the patient's back so they become elevated or raising the operating room table. Note, in this situation, all points would be raised by 1.0 units in y-position. The center of the breast mass 1506 would then take on new coordinates and would be located at (3.0, 3.0). The 4 peri-tumoral sensors/emitters also would take on new coordinates and would be located at (3.0, 5.0) 1508, (1.0, 3.0) 1510, (3.0, 1.5) 1512 and (5.0, 2.0) 1514. The sensor/emitter at the nipple would take on new coordinates of (4.0, 6.8) 1516. Thus, as a patient's position changes, the patient's anatomy takes on new coordinates to account for the change.

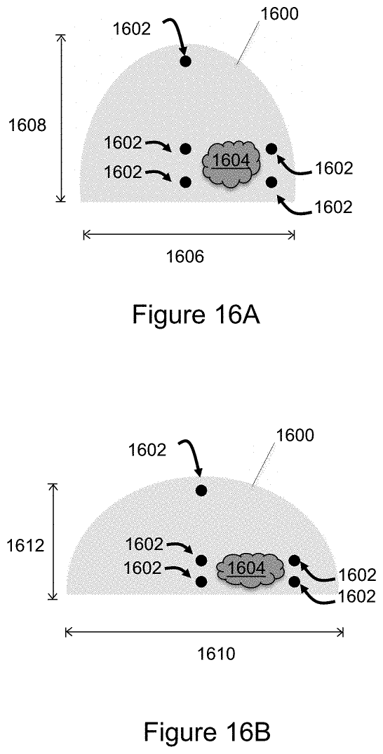

FIGS. 16A and 16B illustrate change in breast configuration with corresponding change in position of sensors/emitters and voxel manipulation to achieve change in configuration of the mass. FIG. 16A illustrates the initial position of the breast 1600, breast mass 1604 and five sensors/emitters 1602. The breast has an initial width 1606 and height 1608. The detectors (not shown) sense the initial position of the sensors and use the information to determine the position, orientation and configuration of the breast mass 1604. FIG. 16B illustrates a change in the patient's breast 1600 configuration and breast mass configuration which is now more flattened. The breast has a different width 1610 and height 1612. The new breast configuration causes the positions of the sensors/emitters 1602 to change correspondingly. The processor performs a voxel manipulation so that mass takes on a new configuration, including changed width and height. The surgeon can see the new configuration and position of the breast mass as a virtual image displayed on the augmented reality headset.

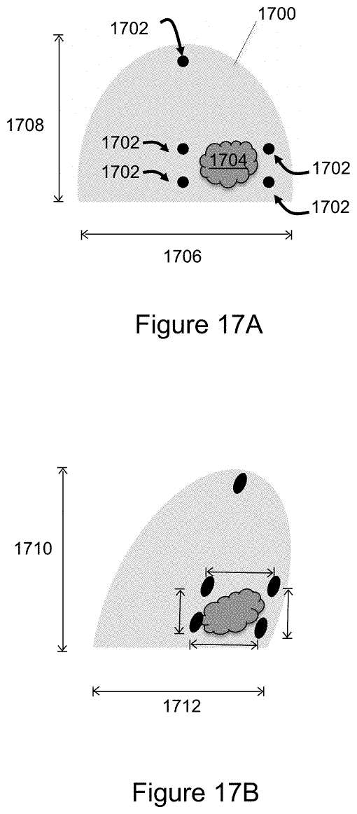

FIGS. 17a and 17B illustrate both a vertical and horizontal change in breast configuration with corresponding change in position of sensors/emitters and voxel manipulation to achieve change in configuration of the mass. For simplicity, only 5 sensors/emitters are shown. In practice, more sensors/emitters might be used. While this figure illustrates position changes in configuration in two dimensions, position changes could also occur in each of three dimensions, e.g. the x-direction, y-direction and z-direction. Also, the breast can undergo rotation (e.g., roll, pitch and yaw). Translation can occur if the breast maintains the same configuration, but moves in position, such as when the operating table being elevated. Also, note that the position (e.g., x-coordinate, y-coordinate, z-coordinate), orientation (i.e., roll, pitch and yaw) and configuration (i.e., shape) of the sensors/emitters can be altered in response to alteration of breast configuration. FIG. 17A illustrates the breast 1700 in its initial configuration with a width 1706 and height 1708. The breast mass 1704 and 5 sensors/emitters 1702 are illustrated in initial locations. FIG. 17B illustrates a change in the breast configuration, which has increased in height 1710 with a slight decrease in width at the base 1712 as if it is being stretched slightly. Note, the position and orientation of the sensors/emitters has also changed. The detectors sense that the position and orientation of the sensors has changed. Note, while this is not shown, the configuration of the sensors/emitters could also change, and this information could be sent via EM signal to the detectors. The processor runs a program and performs a voxel manipulation such that the breast and breast mass and voxels corresponding to the sensor/emitter are adjusted accordingly. The surgeon can view the updated dataset with the voxel manipulations.

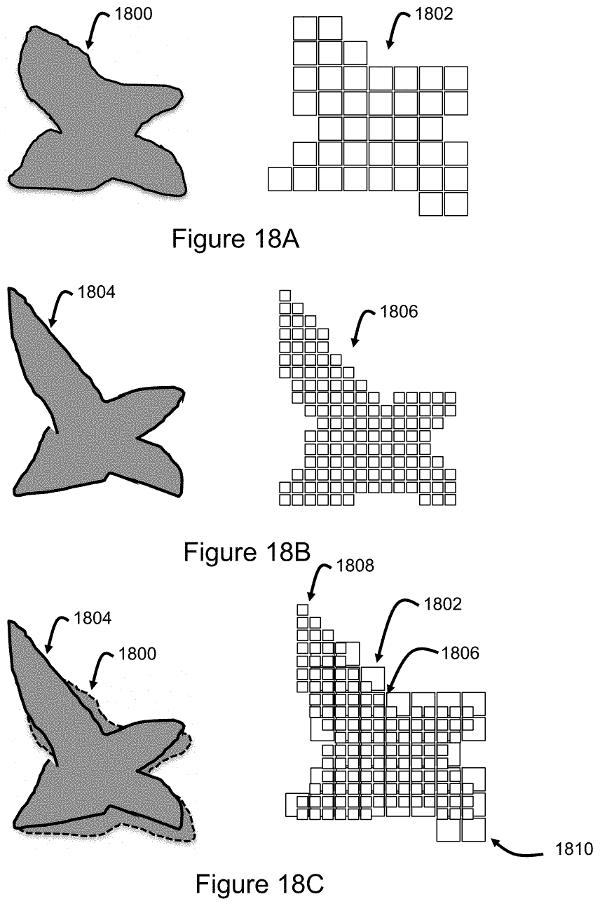

FIGS. 18A through 18C illustrate an example of voxel manipulation performed to better match the shape of the lesion. FIG. 18A illustrates a slice through the actual breast mass 1800 and voxels corresponding to the breast mass 1802. A 2D image is shown, but voxels are 3D entities. FIG. 18B illustrates an altered configuration of the mass 1804. Note that the mass is now slightly taller and narrower. The corresponding voxel manipulation 1806 (i.e., switch to smaller voxels with some voxels being shifted in location) is also shown. Additional options include creation or elimination of voxels. In order to better assess subtle changes in the configuration based on changes in the emitters, one option is to switch to smaller voxels. It should be noted that many types of voxel manipulation are possible as described in U.S. Patent application 62/695,868, titled A VIRTUAL TOOL KIT FOR RADIOLOGISTS, which is incorporated by reference. FIG. 17C illustrates the initial mass configuration and subsequent mass configuration superimposed on one another. Several observations can be noted on the change in configuration of the mass. For example, some voxels 1808 have been created at the top of the lesion to account for stretching of this portion of the mass. Other voxels 1810 have been eliminated.

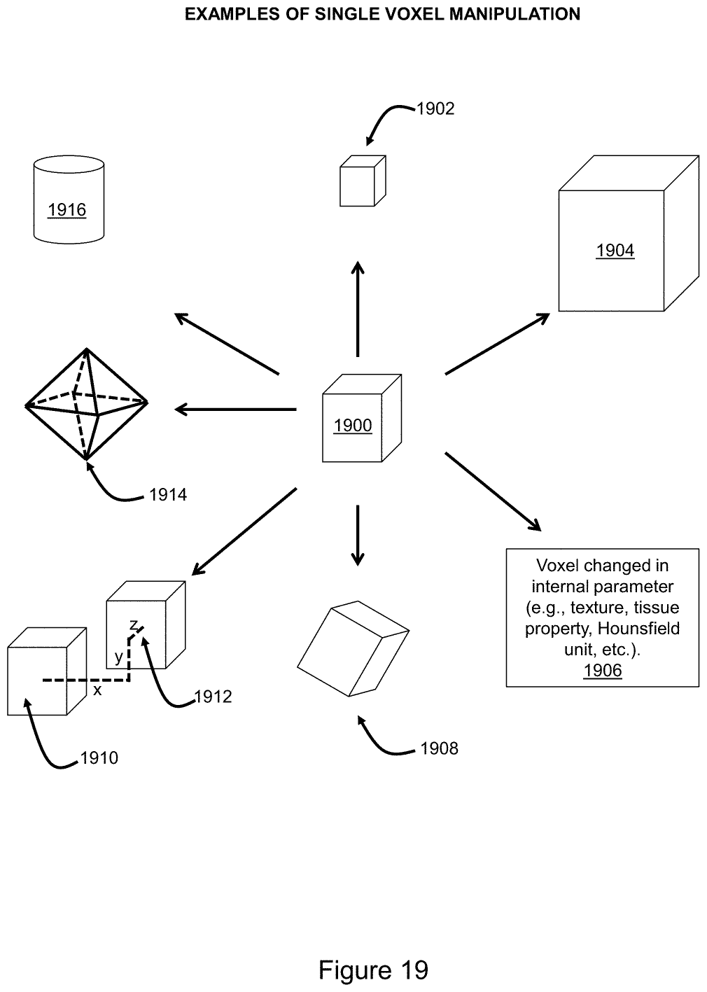

FIG. 19 illustrates examples of a single voxel manipulation with change in voxel size, shape, position, orientation, or internal parameter. It should be noted that unless there is empty space adjacent to the changed voxel, a change in size, shape, orientation or location of the center point of the voxel would impact adjacent voxels. The manner in which the adjacent voxels are impacted is determined by the internal parameter of the adjacent voxels. Since the spatial resolution of many examinations (e.g., CT or MRI) is 1 mm or smaller, small structures can have a poor aesthetic appearance. Take for example, a 3 mm blood vessel making a 180 degree turn over 1 cm. A voxel transformation from cube-shape to cylindrical-shaped voxels would constitute an improvement of visualization. It is anticipated that performing voxel transformations to improve visualization will directly improve diagnosis and patient care. For this illustration the original voxel is a cube shape 1900. The voxel can be manipulated such that it is decreased in size 1902. The voxel can be manipulated such that it is increased in size 1904. The voxel can be manipulated such that the data value 1906 (e.g., tissue property, Hounsfield Unit, etc.) within the voxel is altered. The voxel can be manipulated such that the voxel orientation 1908 (i.e., roll, pitch or yaw) is altered. The voxel can be manipulated such that its location is altered. This can be performed by altering the center point of the voxel in at least one of the x-direction, y-direction or z-direction, such that the center point of the original voxel location 1910 is different from the center point of the final voxel location 1912. The voxel can be manipulated such that its shape is changed such as changing from a cube shape to an octahedron 1914 or changing from a cube shape to a cylinder 1916.

Several features, aspects, embodiments and implementations have been described. Nevertheless, it will be understood that a wide variety of modifications and combinations may be made without departing from the scope of the inventive concepts described herein. Accordingly, those modifications and combinations are within the scope of the following claims.

What is claimed is:

1. An apparatus comprising:
a plurality of radiographically-detectable markers that are implanted in an anatomical structure;
a radiographic scanner that detects the markers in the anatomical structure and generates two-dimensional scans of the anatomical structure; and
an image processor that:
uses the two-dimensional scans to co-register the detected markers with the anatomical structure by calculating a location of each detected marker relative to a location within the anatomical structure;
generates a three-dimensional representation of the anatomical structure based on the two-dimensional scans; and
adjusts the three-dimensional representation of the anatomical structure based on change in location of at least one of the detected markers relative to other ones of the detected markers as indicated in successive two-dimensional scans of the anatomical structure.

2. The apparatus of claim 1 wherein the markers comprise an emitter of electromagnetic energy.

3. The apparatus of claim 1 wherein at least one of the markers comprises a sensor.

4. The apparatus of claim 1 wherein each of the markers comprise a photon-emitting radiopharmaceutical.

5. The apparatus of claim 1 wherein at least some of the markers are interconnected.

6. The apparatus of claim 1 wherein at least some of the markers are attached to a single non-anatomical object.

7. The apparatus of claim 1 wherein each of the makers generates a uniquely identifiable output.

8. The apparatus of claim 1 wherein the image processor adjusts the three-dimensional representation of the anatomical structure by manipulating a plurality of voxels in a coordinated manner.

9. The apparatus of claim 1 wherein the image processor co-registers the location of each detected marker with an operating room coordinate system.

10. The apparatus of claim 1 wherein the image processor calculates a location of a first detected marker based on respective known locations of other ones of the detected markers.

11. The apparatus of claim 1 wherein the image processor adjusts the three-dimensional representation of the anatomical structure based on positional change of at least one of the detected markers.

12. The apparatus of claim 1 wherein the image processor adjusts the three-dimensional representation of the anatomical structure based on orientational change of at least one of the detected markers.

13. The apparatus of claim 1 wherein the image processor adjusts the three-dimensional representation of the anatomical structure based on configurational change of at least one of the detected markers.

14. A method comprising:
implanting a plurality of radiographically-detectable markers in an anatomical structure;
detecting the markers in the anatomical structure;
representing the detected markers in radiological scan of the anatomical structure;
co-registering the detected markers with the anatomical structure by calculating a location of each detected marker relative to a location within the anatomical structure;
generating a three-dimensional representation of the anatomical structure based on the radiological scan; and
adjusting the three-dimensional representation of the anatomical structure based on change of at least one of the detected markers as indicated in real time imaging.

15. The method of claim 14 comprising the markers emitting electromagnetic energy.

16. The method of claim 14 comprising sensing at least one environmental condition of the anatomical structure with at least one of the markers.

17. The method of claim 14 comprising each of the markers using a radiopharmaceutical to emit photons.

18. The method of claim 14 comprising interconnecting at least some of the markers.

19. The method of claim 14 comprising attaching at least some of the markers to a single non-anatomical object.

20. The method of claim 14 comprising each of the makers generating a uniquely identifiable output.

21. The method of claim 14 comprising adjusting the three-dimensional representation of the anatomical structure by manipulating a plurality of voxels in a coordinated manner.

22. The method of claim 14 comprising co-registering the location of each detected marker with an operating room coordinate system.

23. The method of claim 14 comprising calculating a location of a first detected marker based on respective known locations of other ones of the detected markers.

24. The method of claim 14 comprising adjusting the three-dimensional representation of the anatomical structure based on positional change of at least one of the detected markers relative to the other ones of the detected markers.

25. The method of claim 14 comprising adjusting the three-dimensional representation of the anatomical structure based on orientational change of at least one of the detected markers relative to the other ones of the detected markers.

26. The method of claim 14 comprising adjusting the three-dimensional representation of the anatomical structure based on configurational change of at least one of the detected markers relative to the other ones of the detected markers.

27. A method comprising:
a plurality of radiographically-detectable markers that are implanted in a structure;
a radiographic scanner that images the markers and the structure to establish the relationship between the markers and the structure;
a tracking system that continuously updates the position of the markers; and
an image processor that adjusts the three-dimensional representation of the structure based on change in location of at least one of the detected markers.
